(19) United States
(12) Patent Application Publication
(10) Pub. No.: US 2013/0226270 A1
(43) Pub. Date: Aug. 29, 2013

(54) BIOTRANSDUCER FOR TREATING MACULAR DEGENERATION AND OTHER CONDITIONS AND RELATED SYSTEM AND METHOD

(71) Applicant: Jerald L. Tennant, Colleyville, TX (US)

(72) Inventor: Jerald L. Tennant, Colleyville, TX (US)

(21) Appl. No.: 13/776,885

(22) Filed: Feb. 26, 2013

Related U.S. Application Data
(60) Provisional application No. 61/604,068, filed on Feb. 28, 2012.

(51) Int. Cl. A61F 9/007

(52) U.S. Cl. CPC A61F 9/0079
USPC 607/90

(57) ABSTRACT

A system includes a resonant circuit having an inductor coupled to a piezoelectric oscillator. The system also includes one or more light emitting diodes (LEDs) configured to generate illumination based on an output of the resonant circuit. The system further includes a frequency generator configured to frequency modulate the output of the resonant circuit. In addition, the system includes a power supply configured to provide a voltage to the resonant circuit and activate a piezoelectric effect in the piezoelectric oscillator. The piezoelectric oscillator may include a piezoelectric material that is configured to undergo electrical distortion in response to an applied voltage from the power supply. The frequency generator can be configured to alter a capacitance and/or an inductance of the resonant circuit. The illumination can be applied to the retina or other tissue of a body to adjust a pH of cells in the tissue.

### CROSS-REFERENCE TO RELATED APPLICATION AND PRIORITY CLAIM

This application claims priority under 35 U.S.C. §119(e) to U.S. Provisional Patent Application No. 61/604,068 filed on Feb. 28, 2012. This provisional patent application is hereby incorporated by reference.

### TECHNICAL FIELD

This disclosure relates generally to medical devices. More specifically, this disclosure relates to a biotransducer for treating macular degeneration and other conditions and related system and method.

### BACKGROUND

Macular degeneration is a leading cause of blindness in older adults. Macular degeneration affects the central portion of the retina, which is the layer of tissue responsible for converting images into nerve signals at the back of the inner eye. The central portion of the retina is called the “macula.”

There are generally two forms of macular degeneration. “Dry macular degeneration” occurs as the body loses the ability to make new cells to replace damaged cones in the macula. Dry macular degeneration is characterized by yellowish deposits called “drusen” that form to fill the wounds where cones die and are not replaced, pigment clumping of the underlying pigment epithelium, and distorted vision. As oxygen levels drop and more cells die, neovascularization (new blood vessel growth) is stimulated in an attempt by the body to bring oxygen to the area. These new vessels are fragile and can bleed. The vessels themselves distort the macula, further reducing vision. This is called “wet macular degeneration”.

### SUMMARY

This disclosure provides a biotransducer for treating macular degeneration and other conditions and related system and method.

In a first embodiment, an apparatus includes a resonant circuit having an inductor coupled to a piezoelectric oscillator. The apparatus also includes one or more light emitting diodes (LEDs) configured to generate illumination based on an output of the resonant circuit. The apparatus further includes a frequency generator configured to frequency modulate the output of the resonant circuit.

In a second embodiment, a system includes a resonant circuit having an inductor coupled to a piezoelectric oscillator. The system also includes one or more LEDs configured to generate illumination based on an output of the resonant circuit. The system further includes a frequency generator configured to frequency modulate the output of the resonant circuit. In addition, the system includes a power supply configured to provide a voltage to the resonant circuit and activate a piezoelectric effect in the piezoelectric oscillator.

In a third embodiment, a method includes generating a pulsing energy field using a resonant circuit having an inductor coupled to a piezoelectric oscillator. The method also includes generating illumination based on the energy field using one or more LEDs. In addition, the method includes frequency modulating the energy field to modulate the illumination.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 illustrates an example biotransducer in accordance with this disclosure; and

FIG. 2 illustrates an example method for using a biotransducer to treat eye disorders and other conditions in accordance with this disclosure.

### DETAILED DESCRIPTION

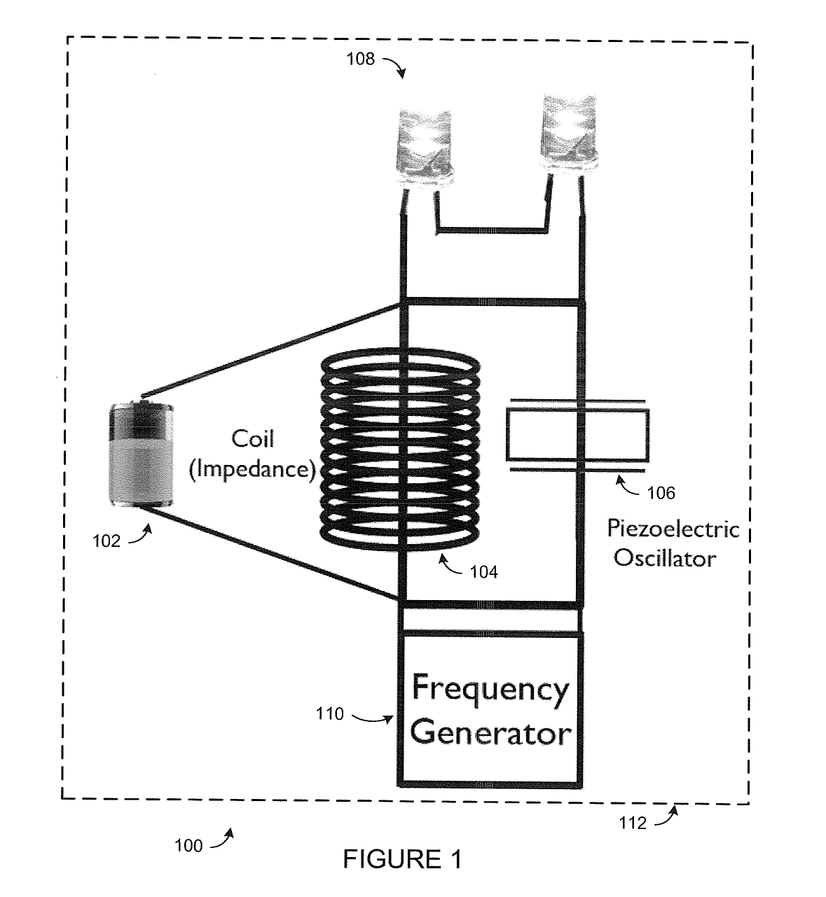

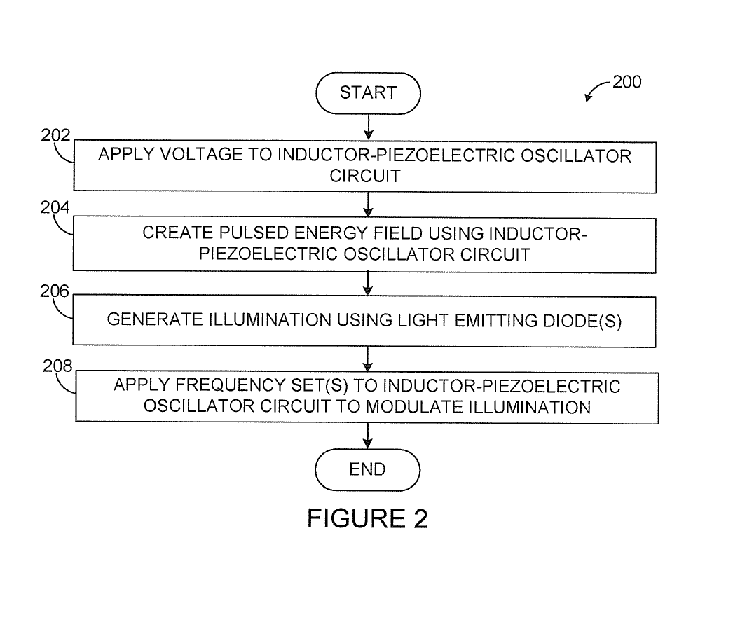

FIGS. 1 and 2, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

The human body maintains and heals itself by making new cells. For example, the retina of the eye (including the macula) replaces itself approximately every three days. Certain illnesses can occur when the body loses its ability to make new cells that work correctly. In addition, cells only function normally in an environment that has the correct pH. For example, various cells are designed to work at a pH of about 7.35 to about 7.45.

A pH level is actually a measurement of voltage. Voltage in a solution can be an electron donor or an electron stealer. If the voltage of a solution is an electron stealer, the voltage is positive. If the voltage of a solution is an electron donor, the voltage is negative. The measured voltage of a solution is converted to a logarithmic scale of 0-14, which represents a measure of pH. A pH value of 7.35 is equivalent to −20 millivolts for an electron donor. A pH value of 7.45 is equivalent to −25 millivolts for an electron donor.

Making new cells may require a certain voltage or voltage range, such as about −50 millivolts. When cells lose their voltage, they may not only lose their ability to function or function efficiently, they can lose the ability replace themselves. As cells begin to malfunction and cannot replace themselves with new cells that have adequate voltage and work normally, this can lead to certain chronic diseases. For example, as the voltage in cells drops, oxygen levels drop because the amount of oxygen that dissolves in a solution is dictated by the voltage of the solution. As oxygen drops, it stimulates the growth of new blood vessels. Early in macular degeneration, there are simply dead or dying cells (dry macular degeneration). As new blood vessels are encouraged to grow, bleeding can occur, further damaging the macula (wet macular degeneration). Macular degeneration occurs when the pH (voltage) of the cells is too low for the cells to function normally and/or to replace themselves with cells that work. This patent document recognizes the role of pH (voltage) as a cause (possibly the primary cause) of chronic diseases including macular degeneration. Clinical experience has shown that when the voltage is corrected in the macula, vision is restored unless there has been too much scarring to overcome.

Almost all current treatments for macular degeneration ignore its basic cause of low pH (voltage) and try to eliminate new blood vessels with lasers or drugs that are intended to shut down these new vessels. In accordance with this disclosure, a biotransducer is disclosed that can adjust the pH (voltage) of cells, such as in the retina, in order to help promote healing.

FIG. 1 illustrates an example biotransducer 100 in accordance with this disclosure. As shown in FIG. 1, the biotransducer 100 includes a power supply 102, which provides energy to other components of the biotransducer 100. The power supply 102 includes any suitable source of power, such as one or more batteries or other DC power source(s) or an AC-to-DC converter.

The power source 102 is coupled across an inductor 104. The inductor 104 is formed by a coil of conductive material. The inductor 104 includes any suitable inductive structure having any suitable inductance.

The inductor 104 is coupled to a piezoelectric oscillator 106. Piezoelectricity is a charge that accumulates in certain solid materials (such as crystals and certain ceramics) in response to applied mechanical stress. The piezoelectric effect is a reversible process in that materials exhibiting the direct piezoelectric effect (the internal generation of electrical charge resulting from an applied mechanical force) also exhibit the reverse piezoelectric effect (the internal generation of a mechanical strain resulting from an applied electrical field). When a piezoelectric material is properly cut and mounted, it can be made to distort in an electric field by applying a voltage to an electrode near or on the piezoelectric material. When the field is removed, the material generates an electric field as it returns to its previous shape, and this can generate a voltage. The piezoelectric oscillator 106 therefore operates to create an electrical signal at a specified resonant frequency. The piezoelectric oscillator 106 could be formed from any suitable piezoelectric material(s), such as a piezoelectric crystal.

In this example, the piezoelectric oscillator 106 takes the place of a capacitor in a typical LC resonant circuit, creating a modified LC-type circuit. The power supply 102 provides a voltage to the modified LC-type circuit to activate the piezoelectric effect in the oscillator 106 by electrical distortion (instead of mechanical distortion). The resulting effect is a pulsing, temporarily self-sustaining energy field with the characteristics of the chosen piezoelectric material and inductor impedance. As can be seen in FIG. 1, the inductor 104 is coupled in parallel with the piezoelectric oscillator 106.

One or more light emitting diodes (LEDs) 108 are arranged to receive an output of the modified LC-type circuit. The LEDs 108 generate illumination at one or more desired wavelengths or in one or more desired wavelength bands. In particular embodiments, the LEDs 108 operate in the range of 380 nm (±20%) to 410 nm (±20%), which is known to affect biological systems. In other particular embodiments, the LEDs 108 operate in the range of 380 nm (±20%) to 900 nm (±20%), which is also known to affect biological systems. Each LED 108 includes any suitable semiconductor structure capable of generating illumination.

A frequency generator 110 is also coupled to the modified LC-type circuit. The frequency generator 110 is capable of applying one or more frequency sets to the modified LC-type circuit to modulate the frequency sets onto the output of the LC-type circuit. This therefore modulates the output of the LEDs 108. The frequency generator 110 can accomplish this in any suitable manner, such as by altering the impedance or capacitance of the modified LC-type circuit. The frequency generator 110 includes any suitable structure for modifying an operating frequency of the modified LC-type circuit.

The components shown in FIG. 1 could be contained within a housing 112. For example, the housing 112 could represent a handheld structure encasing or otherwise carrying the other components 102-110 of the biotransducer 100. The housing 112 can have any suitable size and shape and can be formed from any suitable material(s), such as plastic. The housing 112 may or may not be coupled to external signal or power lines. If external lines are coupled to the housing 112, the external lines could provide operating power, control signals, or other contents to the biotransducer 100. Note, however, that the biotransducer 100 could receive power and/or signals in any other suitable manner, such as via wireless power or signaling.

The biotransducer 100 could be used in any suitable manner. For example, the biotransducer 100 could be placed near a patient's eye so that illumination from the LEDs 108 enters into the patient's eye. This could be done to treat any number of conditions, such as macular degeneration, glaucoma, optic neuritis, uveitis, corneal edema, Fuch's dystrophy, diabetic retinopathy, and other eye diseases. The biotransducer 100 could also be applied to other portions of the body, such as other body organs via the skin or acupuncture meridians. Note that while described as being used to treat humans, the biotransducer 100 could be used to treat any other mammal having a suitable condition.

Although FIG. 1 illustrates one example of a biotransducer 100, various changes may be made to FIG. 1. For example, the biotransducer 100 could include any number of LEDs or other components. Also, the LED(s) 108 could generate illumination at any suitable wavelength(s) or in any suitable wavelength band(s). In addition, note that the components 102-110 could reside in different housings or other structures. As a particular example, the components 102-106 and 110 could reside in a base unit, and the LEDs 108 could reside in a handheld unit that can be positioned to deliver the LED illumination to a patient. As another particular example, the components 102-110 could reside in a base unit, and illumination from the LEDs 108 could be provided to a handheld unit via fiber optic cables or other mechanisms for delivery to a patient.

FIG. 2 illustrates an example method 200 for using a biotransducer to treat eye disorders and other conditions in accordance with this disclosure. The method 200 could, for example, be used with the biotransducer 100 of FIG. 1.

As shown in FIG. 2, a voltage is applied to an inductor-piezoelectric oscillator circuit at step 202. This could include, for example, using the power source 102 to apply a voltage to an inductor-piezoelectric oscillator circuit formed using the inductor 104 and the piezoelectric oscillator 106. This leads to the creation of a pulsing energy field at step 204. For example, the voltage provided by the power supply 102 to the inductor-piezoelectric oscillator circuit activates the piezoelectric effect in the oscillator 106 by electrical distortion, which creates a pulsing, temporarily self-sustaining energy field.

This energy field is applied to one or more LEDs to generate illumination at one or more desired wavelengths or in one or more desired wavelength bands at step 206. This could include, for example, the inductor-piezoelectric oscillator circuit applying the energy field to the LED(s) 108. These LED(s) 108 can generate illumination at the desired wavelength(s) or in the desired wavelength band(s). One or more frequency sets are applied to the inductor-piezoelectric oscillator circuit to modulate the generated illumination at step 208. This could include, for example, the frequency generator 110 applying one or more frequency sets to the inductor-piezoelectric oscillator circuit to modulate the frequency sets onto the output of the inductor-piezoelectric oscillator circuit, thereby modulating the output of the LED(s) 108. This could be done by altering the impedance or capacitance of the inductor-piezoelectric oscillator circuit or in any other suitable manner.

During these steps, the illumination can be directed towards a patient's eye or other organ. The illumination can adjust the pH (voltage) of cells in the patient's body, helping to treat various diseases or other conditions.

Although FIG. 2 illustrates one example of a method 200 for using a biotransducer to treat eye disorders and other conditions, various changes may be made to FIG. 2. For example, while shown as a series of steps, various steps in FIG. 2 could overlap, occur in parallel, occur in a different order, or occur any number of times.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An apparatus comprising:
a resonant circuit comprising an inductor coupled to a piezoelectric oscillator;
one or more light emitting diodes (LEDs) configured to generate illumination based on an output of the resonant circuit; and
a frequency generator configured to frequency modulate the output of the resonant circuit.

2. The apparatus of claim 1, wherein the piezoelectric oscillator comprises a piezoelectric material that is configured to undergo electrical distortion in response to an applied voltage.

3. The apparatus of claim 2, wherein the piezoelectric material is configured to temporarily create a pulsing energy field.

4. The apparatus of claim 1, wherein the frequency generator is configured to frequency modulate the output of the resonant circuit by altering at least one of a capacitance and an inductance of the resonant circuit.

5. The apparatus of claim 1, wherein the one or more LEDs are configured to at least one of:
generate the illumination at one or more desired frequencies; and
generate the illumination in one or more desired wavelength bands.

6. The apparatus of claim 1, wherein the one or more LEDs are configured to generate the illumination between 380 nm (±20%) and 900 nm (±20%).

7. A system comprising:
a resonant circuit comprising an inductor coupled to a piezoelectric oscillator;
one or more light emitting diodes (LEDs) configured to generate illumination based on an output of the resonant circuit;
a frequency generator configured to frequency modulate the output of the resonant circuit; and
a power supply configured to provide a voltage to the resonant circuit and activate a piezoelectric effect in the piezoelectric oscillator.

8. The system of claim 7, wherein the piezoelectric oscillator comprises a piezoelectric material that is configured to undergo electrical distortion in response to an applied voltage from the power supply.

9. The system of claim 8, wherein the piezoelectric material is configured to temporarily create a pulsing energy field.

10. The system of claim 7, wherein the frequency generator is configured to frequency modulate the output of the resonant circuit by altering at least one of a capacitance and an inductance of the resonant circuit.

11. The system of claim 7, wherein the one or more LEDs are configured to at least one of:
generate the illumination at one or more desired frequencies; and
generate the illumination in one or more desired wavelength bands.

12. The system of claim 7, wherein the one or more LEDs are configured to generate the illumination between 380 nm (±20%) and 900 nm (±20%).

13. The system of claim 7, wherein the inductor is coupled in parallel with the piezoelectric oscillator.

14. The system of claim 7, wherein the power supply comprises at least one battery.

15. A method comprising:
generating a pulsing energy field using a resonant circuit comprising an inductor coupled to a piezoelectric oscillator;
generating illumination based on the energy field using one or more light emitting diodes (LEDs); and
frequency modulating the energy field to modulate the illumination.

16. The method of claim 15, wherein:
the piezoelectric oscillator comprises a piezoelectric material that undergoes electrical distortion in response to an applied voltage; and
the piezoelectric material temporarily creates a pulsing energy field.

17. The method of claim 15, wherein frequency modulating the energy field comprises altering at least one of a capacitance and an inductance of the resonant circuit.

18. The method of claim 15, wherein generating the illumination comprises at least one of:
generating the illumination at one or more desired frequencies; and
generating the illumination in one or more desired wavelength bands.

19. The method of claim 15, further comprising:
applying the illumination to tissue of a body to adjust a pH of cells in the tissue.

20. The method of claim 19, wherein the tissue of the body comprises a retina of an eye.
